(19) United States
(12) Patent Application Publication
(10) Pub. No.: US 2008/0208047 A1
(43) Pub. Date: Aug. 28, 2008

(54) Stylus-Aided Touchscreen Control of Ultrasound Imaging Devices

(75) Inventor: Gaspar Delso, Puteaux (FR)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/914,982

(22) PCT Filed: May 12, 2006
(86) PCT No.: PCT/IB2006/051496

§ 371 Date: Nov. 20, 2007

(30) Foreign Application Priority Data
May 25, 2005 (EP) 05300416.4

(51) Int. Cl. A61B 8/00

(52) U.S. Cl. 600/437

(57) ABSTRACT

A system (500), method and device (201-202) as well as an improvement for existing equipment is provided comprising a combination touchscreen/stylus (201-202) control for an ultra sound device such that high-resolution graphical input can be provided, especially concurrent with scanning of a subject. The ultra sound device can be controlled with only the provided touchscreen/stylus (201-202), or traditional trackball, mouse and keyboard input devices can also be provided for non-graphical input. An implementation approach that utilizes existing software for processing stylus input is also provided as an improvement to existing ultra sound devices.

The present invention relates to an apparatus, system and method for a stylus and a touchscreen for high resolution, graphical primitive-based user control of an ultra sound (US) imaging device. More particularly, the present invention provides a stylus and touchscreen as a high resolution, graphical primitive-based user control device at least during the scanning operation of an ultra sound (US) imaging device.

Medical ultra sound (US) imaging devices require a user to hold a scanner in one hand while performing a scan of a subject. The number and complexity of control functions that can be performed while scanning with a scanner held in one hand is limited by the dexterity of the technician performing the scan and the type of user interface provided to the other hand of the technician. More particularly, the resolution of the input device dictates the type of control functions that can be performed during the scanning operation of a US device.

The use of a stylus (a pointed instrument used as an input device on a pressure/touch-sensitive screen) as a means to interact with personal digital assistants (PDA) and other hand-held devices is a common, widely spread practice and is known for US imaging device control.

An input/output system incorporating a handheld imaging device and having at least one input/output device comprising a touchscreen sensitive to the pressure of a finger or stylus, and to a voltage or current produced by a stylus, has been disclosed in U.S. Patent Application No. 20040138569 by Grunwald, et. al., the entire contents of which are hereby incorporated by reference herein. Grunwald's teaching, however, uses the stylus in a two-handed operation with one hand controlling the stylus while the other hand manipulates a set of tactile controls. Grunwald's teaching does not apply to using the stylus while scanning to control an ultra sound (US) imaging device. Grunwald's teaching uses a stylus only when not holding a scanner. Further, Grunwald does not teach graphical primitive-based control input via the stylus.

U.S. Pat. No. 6,638,223 to Lifshitz et. al., teaches an ultra sound imaging device comprising a touchscreen, disposed in front of a monitor for producing an image display and having activation areas pre-assigned to specific functions so that no external input other than that supported by the touchscreen is required to operate the ultra sound (US) imaging device. A processor coupled to the touchscreen detects a touch in one of the pre-assigned activation areas and performs the function associated with that activation area. The functions include those required to implement an ultra sound (US) diagnostic system and are implemented by function modules comprising a function set of system software (col. 2, line 59 et seq.). Lifshitz's teaching uses a finger, pen, or other pointer to “touch” the touchscreen (col. 5, lines 21-22). While Lifshitz teaches that an activation area may be defined in location and size by absolute pixel regions (col. 5, line 31 et. seq.), Lifshitz does not teach high resolution graphical primitive-based input to control the US device via a stylus.

One advantage cited for the use of touchscreens is to reduce clutter in the US control area, see, e.g., U.S. Patent Application No. 20040179332 to Smith et. al. Typically, as Smith discloses, a hierarchy of menus is navigated using some type of pointing device with inactive screens being hidden from view or displayed in some way that indicates to the user that these screens are currently inactive (e.g., by changing their color). In such a touchscreen and pointing device scenario, high resolution input is not required nor is it disclosed in the prior art. On-screen keyboards have also been suggested and/or provided for input of patient data and annotations, but they do not satisfactorily replace a standard keyboard for input of any but small amounts of text.

Further, clinical ultrasonography practice requires some kind of interaction with the as-acquired data. This interaction ranges from the simple definition of a region of interest (ROI) to the initialization of sophisticated image processing algorithms. As medical image processing evolves, more and more input must be provided often in the form of graphical primitive input: Points, segments, lines and planes must be defined; in two or three-dimensional space, complex curves must be drawn on the acquired data. Current ultra sound machines are equipped with input devices that either provide low resolution input or are inadequate to perform complex tasks during a US scan, such as the outlining of the ventricular cavity or the trimming of undesired tissue in a 3-dimensional fetal scan. For example, a mouse input device is ill adapted to the ultrasonography environment (e.g., the mouse device must be held with the left hand, as the right hand is used to hold the US probe, a flat surface for deploying the mouse is not always available, US gel tends to get in the mouse).

A high resolution device is thus needed for control of a US imaging device, especially during the scanning operation of the US imaging device. The system and method of the present invention provides high-resolution stylus-based input and touchscreen to control a US imaging device.

In one preferred embodiment, high-resolution stylus interaction using a touchscreen is an adjunct to a mouse, trackball and button/slider/dial-based operation. In an alternative preferred embodiment the touchscreen and stylus combination is the only user input means and is further supported with a set of lower-resolution touchscreen-activatable software button menus, sliders and dials that are selectable/movable with the stylus by touching the screen for a button or sliding/rotating the stylus across/around, respectively, and a displayed ruler/dial.

High resolution input of the present invention enables the use of an increasing set of interactive graphics-based tools and thus enhances medical practice. Further, with the reduction of hardware in one preferred embodiment (i.e., no mouse, trackball, or sliders) resulting from the touchscreen/stylus-only control combination, comes an additional reduction in control software complexity and an increase is robustness of the US imaging device. Far fewer interfaces need to be provided and supported, and those that are provided are standardized so that upgrades are more easily accomplished. Trouble-shooting is also reduced in this embodiment as is training and the likelihood of user error over the prior art in which errors due to a multiplicity of many-handed control devices are eliminated.

In summary, the most important advantage provided by the high resolution graphical primitive-based stylus-based control for a US imaging device of the present invention lies in the increase in accuracy and versatility of input provided by the stylus and touchscreen, which, coupled with the real-time graphical primitive reinterpretation of this input made possible by this approach, enables an increased set of interactive graphical tools and image processing algorithms that are not practical or even feasible using standard stylus input. Examples of interactive graphical tools made possible with the present invention include
1. defining and managing seed points in two and three dimensions;
2. defining and steering cutting lines and planes; and
3. defining and managing curves either by delineation or control point placement.
   Examples of image processing algorithms enabled by the interaction of the present invention include:
1. single-click automatic alignment of oriented 3d data;
2. curve-initialized 2d/3d segmentation of the left ventricle;
3. curve-initialized 2d/3d fetal abdominal volume estimation; and
4. 2d/3d Doppler-based measurement of blood flow through at least one of a line and a surface.
   Other advantages of the dedicated graphical reinterpretation made possible by the present invention include:
1. reduction of required storage and processing of extraneous and irrelevant input data;
2. increase in the accuracy of user interaction; and
3. reduction of the time required for ultra sound procedures such as point and region-of-interest selection, cutting line/plane definition and editing, image annotation, structure delineation, etc.

Finally, the advantages of stylus interaction accrue to the system and method of the present invention, namely:
1. minimal training is required;
2. it is readily performed with the other hand than that manipulating a scanning head; and
3. due to the passive nature of the device, it is insensitive to ultra sound gel and other products found in clinical practice.

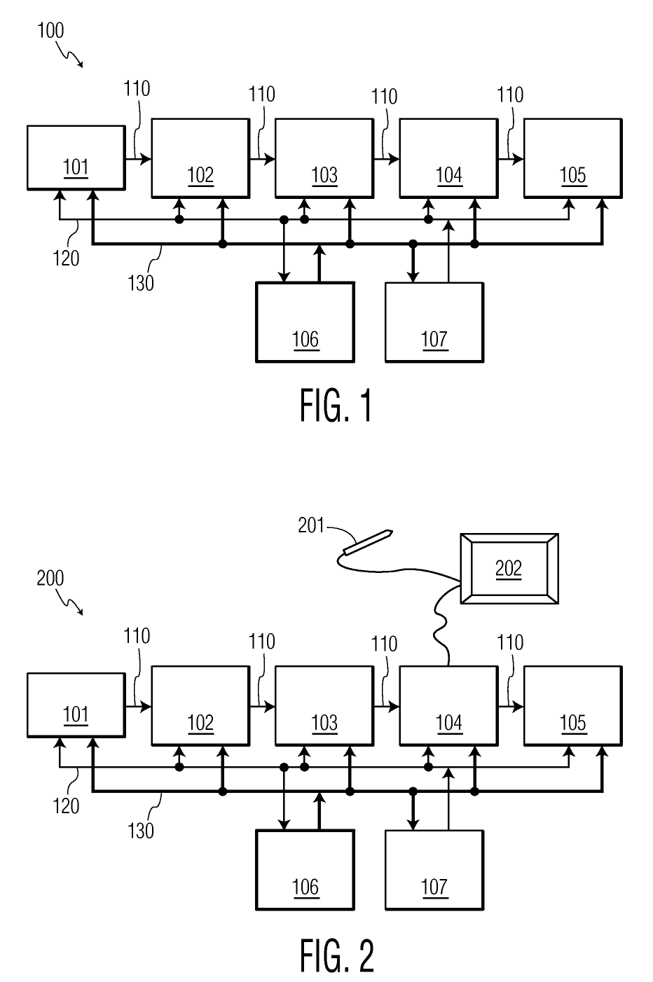

FIG. 1 illustrates a generic configuration of a US imaging device;

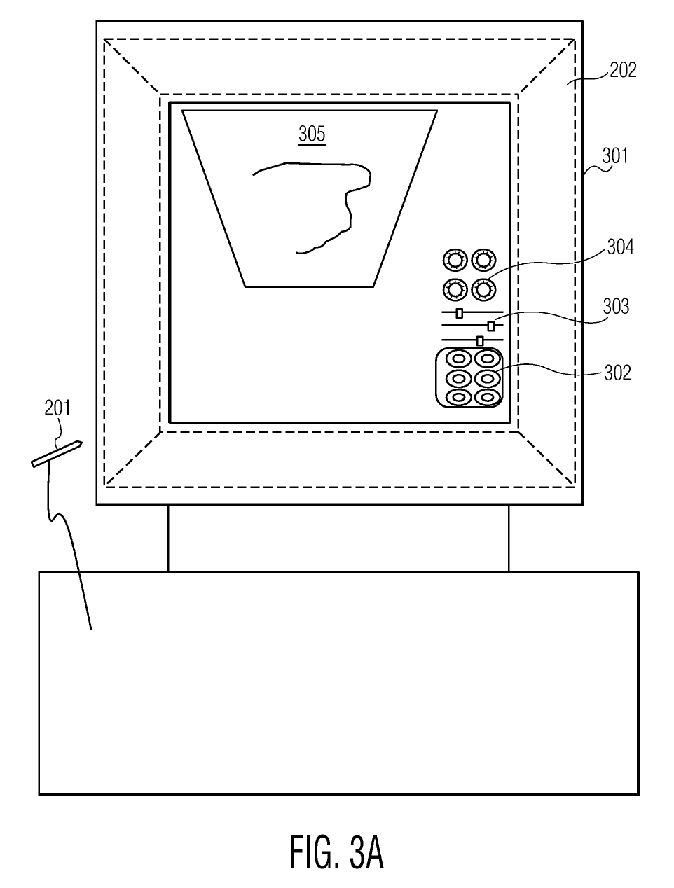

FIG. 2 illustrates a generic US configuration modified according to the present invention;

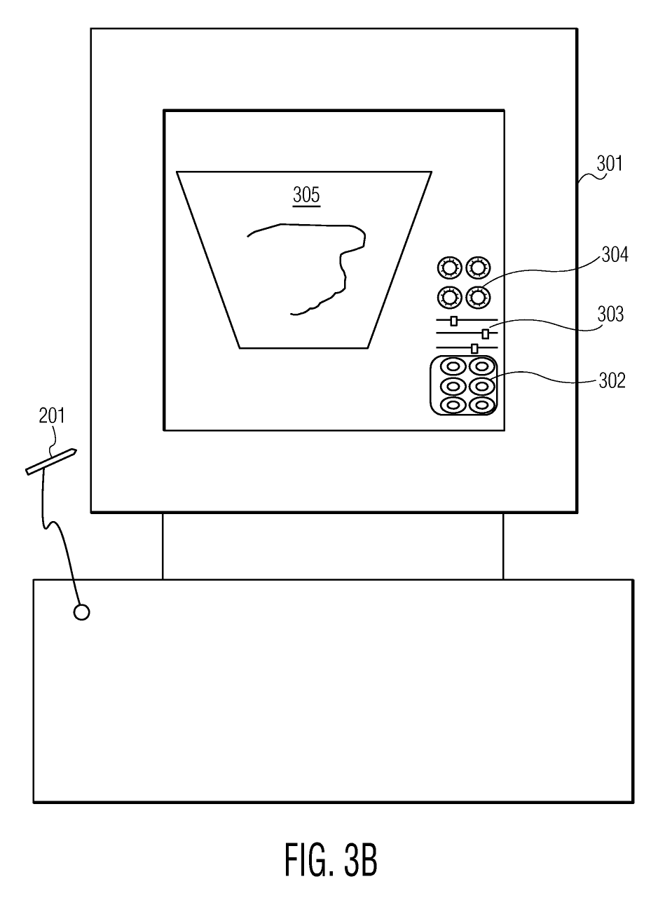

FIG. 3A illustrates a generic US device with a touchscreen/stylus modification according to the present invention having the touchscreen placed above the generic system's screen;

FIG. 3B illustrates a US device with a touchscreen/stylus for user input according to the present invention;

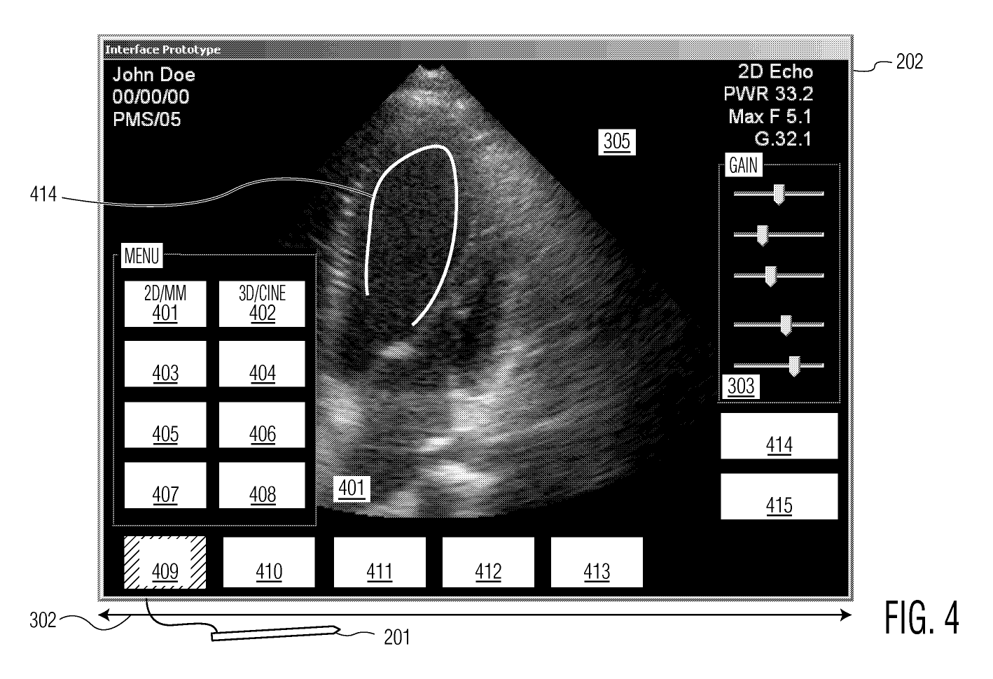

FIG. 4 illustrates a spline curve drawn with a stylus and the placement of soft input buttons and sliders in a touchscreen having a stylus with the display of a concurrently scanned US image;

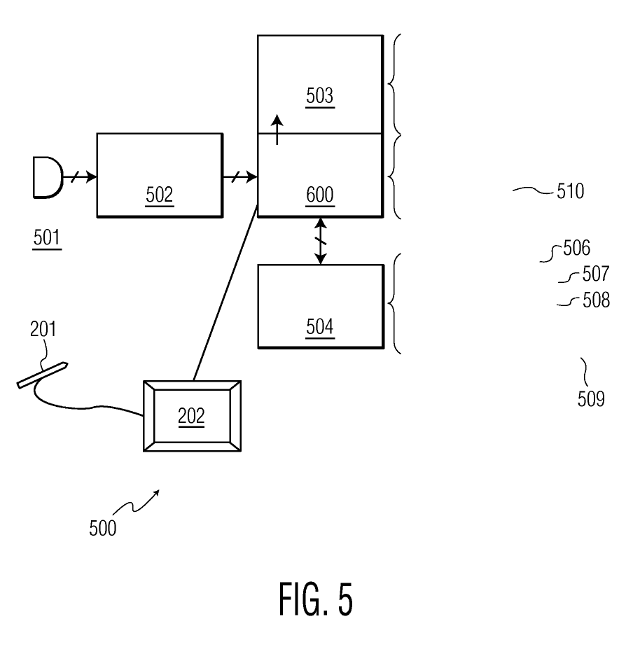

FIG. 5 illustrates US system task distribution according to an embodiment of the present invention; and

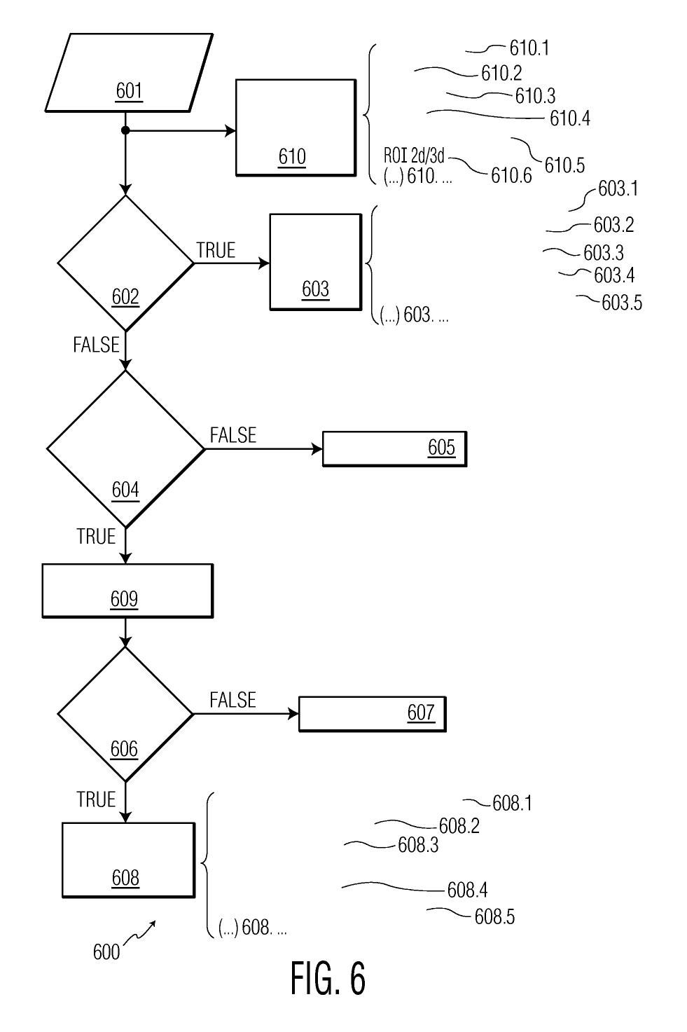

FIG. 6 illustrates a processing flow for stylus events that occurs concurrent with scanning, according to the present invention.

It is to be understood by persons of ordinary skill in the art that the following descriptions are provided for purposes of illustration and not for limitation. An artisan understands that there are many variations that lie within the spirit of the invention and the scope of the appended claims. Unnecessary detail of known functions and operations may be omitted from the current description so as not to obscure the present invention.

FIG. 1 illustrates a configuration for a typical US imaging system. A scanner or transducer 101 transmits sound waves acquired by acquisition subsystem 102. The acquired sound waves are subjected to signal processing by subsystem 103 and then displayed by display subsystem 104. Interfacing with the user and other systems, such as a database and network, is made through interface subsystem 105. User input is captured from devices such as a traditional mouse and track-ball and the stylus and/or touchscreen of the present invention. Control subsystem 106 is in charge of monitoring, synchronizing and managing the whole ultra sound system operation. Power is supplied to the various subsystems by a power supply 107 and the various subsystems are connected to one another, typically through a system bus 130 as shown in FIG. 1.

In a preferred embodiment, as illustrated in FIG. 2, a stylus 201 optionally connected to the power supply 107 must be included as in an input device to a touchscreen that must be included in the interface subsystem 105.

As most modern ultra sound machines do already include touch-sensitive screens, the hardware modifications required to add a high-resolution stylus interaction are minimal. Further, as illustrated in FIG. 2, minimal modifications to the software are required. In particular, the user interface subsystem is to be adjusted to include a stylus trajectory input stream and stylus contact on or near displayed buttons, sliders and dials presented for selection using a touch-sensitive display. In the present invention the touch sensitive display 202 can be separate from and placed above another display or can be physically integrated with the display that presents images, e.g., US images, and US control elements, e.g., displayed buttons 302, dials 304, and sliders 303, see FIGS. 3A and 3B.

Unlike any existing ultra sound system, the touchscreen/stylus input of the present invention is directly linked to a graphical interpretation device that can take the shape of dedicated hardware or be included as an independent procedure 600 in the processor 503 in charge of the interface operations. This graphical interpretation module 600 is in charge of continuously translating the user input into equivalent graphical primitives such as points in two and three dimensions, segments, lines and planes, as well as making these estimates evolve as new input is received.

Referring now to FIG. 4, the stylus of the present invention enables on-the-touchscreen button 302, dial 304, slider 303 and other input selection at a gross level, e.g., using the stylus 201 as a pointing device to point proximate to a desired button 302, move a slider 303, rotate a dial 304, select from a drop down list, etc., and without switching input devices or mode of US operation enables the use of the same input device (stylus 201) to provide higher-resolution pixel selection of seed points and curves, such as the spline curve 414 delineating the endocardium in FIG. 4.

Consider as an example a US application of ovarian cyst border detection. One image enhancement technique is seed-based region growing in which a pixel in a region of interest is used as a seed point. In the system and method of the present invention the accurate selection of such a seed point is possible, given the high resolution input (pixel level resolution) resulting from the use of the touchscreen and stylus. This input is immediately reinterpreted as a point in data space using the appropriate information about the origin, pixel dimensions and display properties. Subsequent to selection of a pixel as the seed point using the stylus, an image processing algorithm is applied to all similar points (spatially close pixels sharing the same features) are gathered together in the same region. A gray scale difference is one region-growing method. Defining the ROI is one of the most important steps in characterizing tissue because it forms the basis for all subsequent steps. Next, given the seed point, one approach defines a local rectangular seed region centered at the seed point in accordance with a pre-defined homogeneity criterion. The seed region is contracted until one is obtained that satisfies the pre-determined homogeneity criterion. Given this seed region, it is grown by thin adjacent side rectangles using a statistical measure of the side region and a threshold condition to determine statistical similarity until no adjacent rectangles can be found. In this way the edge of an ovarian cyst is determined that depends on the proper selection of a seed point. The touchscreen and stylus of the present invention enable pixel-level seed point selection during the scanning operation of the US imaging device which allows the refinement of US images gathered based on real-time feedback to the operator of the US device without requiring the operator to switch from scanning to non-scanning modes. The edge of the cyst can then be further refined by translating it into a parametric curve model, a graphical primitive which the user can manipulate by simple stylus interaction.

Thus, the high resolution input of the stylus/touchscreen combination of the present invention not only allows the boundary surface detection process to be conducted quickly during a scan, but also enables a more accurate selection by the US operator of the single seed point in the first place and allows a quick correction of the results based on graphical primitive editing.

Consider as another example of primitive-based interaction the cutting of 2d/3d data along lines and planes respectively. In this case, the continuous reinterpretation of the stylus trajectory as either a line or a plane primitive is used to generate an appropriate view of the ultra sound data. A smooth variation of the cutting line or plane would be possible within the described system, and impossible within the scope of the existing patents.

Consider as another example of primitive-based interaction the initialization of a segmentation of the endocardium by means of a user-defined curve. Classical delineation would be either impossible or impractical with the existing systems, whereas stylus interaction considerably simplifies such a task. Furthermore, the fact of reinterpreting such input as a parametric curve primitive enables easy editing of both the delineation and segmentation results, which would not be possible within the scope of the existing patents.

Referring again to FIG. 4, the control functions that can be performed during scanning with the touchscreen/stylus combination of the present invention include, among others:
1. depth gain control;
2. focus control;
3. doppler gate placement and steering 405;
4. m-mode line definition; and
5. feature highlighting and annotation (as in breast imaging).
   The control functions performed after scanning include:
1. automatic and semi-automatic 2D 401 and 3D 402 segmentation (as in cardiac wall segmentation);
2. automatic clutter removal (as in 3D fetal imaging); and
3. automatic feature view optimization 415 (as in fetal imaging).

With regard to a preferred software implementation of the system and method of the present invention, FIG. 5 illustrates a typical organization of typical US software modules. A processor 503 manages the US system comprising a US scanhead or probe 501 for emitting and capturing US signals, front-end signal processing hardware 502 which, in an alternative embodiment, further comprises data processing capabilities (e.g., a separate data processing facility or connection thereto 504, possibly via a network, all not shown). The processor 503 controls input/output operations, which include translation from user input into internal parameter settings and it is at the processor level that all necessary software for stylus 201 control is provided, in a preferred embodiment. In a preferred embodiment for modifying existing US imaging devices, stylus control software 600 is incorporated into the processor 503 by modifying existing I/O handlers to accept the input provided by the stylus 201 of the present invention. Specific software is included at this point to generate, as input arrives, the whole range of graphical primitives 610 that are needed by the system and method of the present invention. Such primitives may include, among others:
1. points in two and three-dimensional space 610.1;
2. lines 610.2;
3. segments 610.3;
4. planes 610.4;
5. parametric curves 610.5; and
6. regions of interest in two and three dimensions 610.6.

In an alternative embodiment this can be done by some specific hardware directly connected to a stylus/touchscreen 201-202 controller (not shown).

In new US devices providing only a touchscreen/stylus 201-202 combination for control, the arrangement is the same as that illustrated in FIG. 5 and does not include software for such devices as a trackball or a mouse. In a preferred embodiment for such a configuration, an on-the-touchscreen keyboard (not shown) replaces a traditional keyboard for inputting annotations and patient information.

FIG. 6 illustrates the software processing flow 600 performed by a host 503 for a US device providing hand-held stylus input concurrent with scanning a subject. As illustrated in FIG. 6, a typical workflow in response to a stylus proximity event includes queuing, for subsequent processing, stylus inputs in an interaction queue 601. Each queued event is removed from the queue according to a pre-determined scheme and the type of proximity event (low resolution) is determined at steps 602 and 604. If the event is within a pre-determined tolerance of a soft button, dial, slider, etc., then the associate event handler is called at step 603. These events include:
1. change menu environment 603.1;
2. start/freeze/stop scan 603.2;
3. change scan modality 603.3;
4. change scan parameters 603.4; and
5. change display parameter 603.5.

In a preferred embodiment, if the stylus input event is a high-resolution graphical input, it must be expected at step 604 or it is ignored at step 605. In an alternative embodiment (not illustrated) a graphical input event automatically causes a switch to processing of such events until a soft button event occurs. When graphical input is valid, at step 606 the input value is validated as being within a pre-determined range and if not valid is ignored at step 607, whereas, if valid, an appropriate routine is invoked at step 608, which routines can include:
1. graphical primitive interpretation 608.1;
2. 2D/3D display control 608.2;
3. view optimization 608.3;
4. assisted/automated segmentation and quantization 608.4; and
5. data annotation and tagging 608.5.
   That is, graphical input can include single and multiple point input, cut plane definition, curve delineation, text handwriting and any other input not explicitly covered by a soft button, slider, and dial.

The present invention is applicable to any ultra sound scanner capable of hosting a touch screen. Within medical practice, the fields of application that benefit from easier interfacing with the present invention range from cardiology to gynecology and obstetrics.

While the preferred embodiments of the present invention have been illustrated and described, it will be understood by those skilled in the art, the US device architecture and methods as described herein are illustrative and various changes and modifications may be made and equivalents may be substituted for elements thereof without departing from the true scope of the present invention. In addition, many modifications may be made to adapt the teachings of the present invention to a particular situation without departing from its central scope. Therefore, it is intended that the present invention not be limited to the particular embodiments disclosed as the best mode contemplated for carrying out the present invention, but that the present invention include all embodiments falling with the scope of the appended claims.

1. A system (100) for user direction of the operation of an ultra sound imaging device (301), comprising:
a touchscreen (202) configured with respect to an image (305) produced by said ultra sound device (301) and operative to detect thereon high and low resolution image-associated user input and low resolution control-associated user input from a user input device;
a stylus (201) to provide said user input as said high and low resolution input and coupled to said touchscreen (202) as said user input device;
a processor (503) configured to accept said high and low resolution user input from said stylus (201) and based on said high and low resolution user input to direct processing thereof by:
at least one of a plurality of graphical input handlers (608) to process said high and low resolution image-associated input; and
at least one of a plurality of control event handlers (603) to process said low resolution control-associated input.

2. The system of claim 1, wherein:
said processor (503) further comprises a stylus module (600) to perform stylus input processing (510) of said high and low resolution stylus input, and based on said stylus input processing generate graphical primitives for processing by said plurality of graphical input handlers (608); and
further comprising a data-processing component (504) interfaced to said stylus module (600) to perform image processing including preprocessing (506), scan conversion (507), post processing (508) and applying high-resolution image processing algorithms (509).

3. The system of claim 2, wherein said high-resolution image processing algorithms (509) include single-click automatic alignment of oriented 3D data, curve-initialized 2D/3D segmentation of the left ventricle, curve-initialized 2D/3D fetal abdominal volume estimation, and 2D/3D Doppler-based measurement of blood flow through at least one of a line and a surface.

4. The system of claim 1, wherein said plurality of control event handlers (603) comprises handlers for a change menu environment (603.1), a start/freeze/stop scan (603.2), a change scan modality (603.3), a change scan parameters (603.4), and a change display parameters (603.5).

5. The system of claim 4, wherein:
said processor (503) further comprises a stylus module (600) to perform stylus input processing (510), and based on said input processing generate graphical primitives for processing by said plurality of graphical input handlers (608); and
further comprising a data processing component (504) interfaced to said stylus module (600) to perform image processing including preprocessing (506), scan conversion (507), post processing (508) and applying high-resolution image processing algorithms (509).

6. The system of claim 5, wherein said high-resolution image processing algorithms (509) include single-click automatic alignment of oriented 3D data, curve-initialized 2D/3D segmentation of the left ventricle, curve-initialized 2D/3D fetal abdominal volume estimation, and 2D/3D Doppler-based measurement of blood flow through at least one of a line and a surface.

7. The system of claim 5, wherein said stylus input processing (510) and said graphical input handlers (608) together implement graphical interactive tools including seed point definition and management in two and three dimensions, cutting line and plane steering and definition, and curve definition and management using delineation and control point placement.

8. The system of claim 1, wherein said stylus (201) and touchscreen (202) form a combination that is configured as an adjunct to at least one other user input device selected from the group consisting of a mouse, a trackball, at least one button, at least one slider and at least one dial.

9. The system of claim 1, wherein:
said stylus (201) and touchscreen (202) form a combination constituting the only user input device; and
said touchscreen (202) further comprises at least one displayed lower-resolution and touchscreen-activatible input feature (302-304) selected from the group consisting of software button menu (302), software slider (303), software dial (304), and on-the-touchscreen keyboard for inputting annotations and patient information wherein said input feature (302-304) is one of selectable and movable by touching the touchscreen (202) with said stylus (201) thereby selecting a button of said button menu (302), sliding a stylus (201) across said slider (303), rotating said stylus (201) around said dial (304) and selecting a key of said on-screen keyboard.

10. The system of claim 1, wherein said user input from said stylus (201) and processing thereof by said processor (503) occurs both simultaneously with scanning and separately after scanning, scanning being a real-time acquisition and a display of ultra sound images by said ultra sound imaging.

11. The system of claim 10, wherein said plurality of graphical event handlers (608) comprises a graphical primitive interpreter (608.1), a 2D/3D display control (608.2), a view optimization (608.3), an assisted/automated segmentation and quantization (608.4), and a data annotation and tagging (608.5).

12. An improvement to an ultra sound device, wherein said improvement comprises:
a touchscreen/stylus combination (201-202) input device to provide high and low resolution user input; and
a stylus input processing module (600) that accepts user input provided by the stylus/touchscreen combination (201-202) and generates therefrom a plurality of high-resolution graphical primitives (610) including points (610.1), lines (610.2), segments (610.3), planes (610.4), parametric curves (610.5), and regions of interest (610.6) in two and three dimensions, and wherein said stylus input processing module (600) also converts user stylus inputs for input to existing ultra sound user input processing capabilities.

13. The ultra sound device of claim 12, wherein said stylus input processing module (600) is a specific hardware component directly connected to the stylus/touchscreen combination (201-202) to generate, as stylus input arrives, said plurality of graphical primitives (610) and inputs to existing ultra sound user input processing capabilities.

14. The ultra sound device of claim 13, further comprising an on-the-touchscreen keyboard for inputting ultra sound image annotations and associated patient information.

15. A method for directing the operation of an ultra sound imaging device, comprising the steps of:
configuring a touchscreen (201) with respect to a displayed ultra sound image (305) of said imaging device to detect thereon (201) high and low resolution image-associated user input and low resolution control-associated user input from a user input device;
coupling a stylus (202) to said configured touchscreen (201) as said user input device to provide high and low resolution user input;
receiving said high and low resolution stylus input by said processor (503);
processing said high and low resolution image-associated user input by a plurality of graphical input handlers (608); and
processing said received low resolution control-associated user input by a plurality of control event handlers (603).

16. The method of claim 15, wherein:
said step of processing by a plurality of graphical input handlers further comprises the step of first generating graphical primitives (610) from said high and low resolution image-associated user input; and
said step further comprises the steps of performing data processing of said high and low resolution stylus input including the substeps of:
preprocessing (506);
scan converting (507);
post processing (508); and
applying high-resolution image processing algorithms (509).

17. The method of claim 15, further comprising the step of:
displaying for selection by said stylus and detection by said touchscreen, a plurality of low resolution input features (302-304) selected from the group consisting of software button menu (302), software slider (303), software dial (304), and on-the-touchscreen keyboard; and
wherein, the steps of configuring the touchscreen and coupling said stylus (201) thereto, and displaying low resolution input features result in forming a combination constituting the only user input device for directing the operation of the ultra sound imaging device.

18. The method of claim 15, wherein the coupling step further comprises coupling said stylus (202) to said configured touchscreen (201) as the only said user input device to provide high and low resolution user input.

19. A device (201-202) for low and high resolution input of user control information for an ultra sound device, comprising:
a touchscreen (202) configured with respect to an image (305) produced by said ultra sound device (301) and operative to detect thereon high and low resolution image-associated user input and low resolution control-associated user input from a user input device;
a stylus (201) to provide said user input as said high and low resolution input and coupled to said touchscreen (202) as said user input device;
a stylus module (600) to perform stylus input processing (510) of said high and low resolution stylus input, and based on said stylus input processing generate output comprising graphical primitives and other image-associated data for processing by a plurality of graphical input handlers (608) and low resolution control-associated output for processing by a plurality of control event handlers (603).

20. The device (201-202) of claim 19, wherein the stylus coupled to the touchscreen is the only user input device for controlling the operation of the ultra sound imaging device.
